(19) United States
(12) Patent Application Publication
(10) Pub. No.: US 2018/0092577 A1
(43) Pub. Date: Apr. 5, 2018

(54) SYSTEMS AND METHODS FOR DETERMINING AN ABNORMAL GLYCEMIC EVENT USING SURROGATES FOR GLUCOSE

(71) Applicant: Cardiac Pacemakers, Inc., St. Paul, MN (US)

(72) Inventors: Michael J. Kane, St. Paul, MN (US); Keith R. Maile, New Brighton, MN (US)

(21) Appl. No.: 15/687,439

(22) Filed: Aug. 26, 2017

Related U.S. Application Data
(60) Provisional application No. 62/380,259, filed on Aug. 26, 2016.

(51) Int. Cl. A61B 5/145; A61B 5/1459; A61B 5/0205; A61B 5/04; A61B 5/0468; A61B 5/16; A61B 5/07

(52) U.S. Cl. CPC A61B 5/14532; A61B 5/1459; A61B 5/1451; A61B 5/0205; A61B 5/082; A61B 5/0468; A61B 5/16; A61B 5/14546; A61B 5/076; A61B 5/04001

(57) ABSTRACT

Systems and methods for determining an abnormal glycemic event using surrogates for glucose are disclosed herein. In an embodiment, a medical system includes a medical device associated with a subject and a processor communicatively coupled to the medical device. The medical device is configured to sense a signal corresponding to a presence of a compound in at least one of: an exhalation breath, interstitial fluid, blood and urine, wherein the compound is a surrogate for glucose. The processor is configured to receive the signal corresponding to the presence of the compound; determine the presence of the compound based on the received signal; and determine the subject is experiencing an abnormal glycemic event in response to the determined presence of the compound.

### CROSS-REFERENCE TO RELATED APPLICATION

This application claims priority to Provisional Application No. 62/380,259, filed Aug. 26, 2016, which is herein incorporated by reference in its entirety.

### TECHNICAL FIELD

Embodiments of the present disclosure relate to systems and methods for determining abnormal glycemic events. More specifically, embodiments of the disclosure relate to systems and methods for determining abnormal glycemic events using compounds that are surrogates for glucose.

### BACKGROUND

Conventionally, an abnormal glycemic event is determined by measuring a subject's glucose levels. However, many conventional systems and methods that measure glucose levels are oftentimes transient in time. For example, subjects may be required to prick their fingers to measure their glucose levels. Many subjects that are required to prick their fingers, however, may either forget to check their glucose levels and/or not check their glucose levels often enough to prevent a hyperglycemic event or a hypoglycemic event. As such, many subjects may experience hyperglycemic events or hypoglycemic events when their blood sugar gets too high or too low, respectively.

Systems and methods that have been designed to measure glucose more frequently, however, may have other shortcomings. For example, these systems and methods may only be effective for a short period of time (e.g., on the order of a week) and/or may be inaccurate due to the difficultly of measuring glucose in vivo. Accordingly, there is a need in the art for alternative systems and methods for determining an abnormal glycemic event.

### SUMMARY

In an Example 1, a medical system comprises: a medical device associated with a subject, wherein the medical device is configured to sense a signal corresponding to a presence of a compound in at least one of: an exhalation breath, interstitial fluid, blood and urine, wherein the compound is a surrogate for glucose; and a processor communicatively coupled to the medical device, the processor configured to: receive the signal corresponding to the presence of the compound; determine the presence of the compound based on the received signal; and determine the subject is experiencing an abnormal glycemic event in response to the determined presence of the compound.

In an Example 2, the medical system of Example 1, wherein the medical device is an implantable medical device and comprises an indicator tag, wherein the indicator tag is responsive to the compound; and wherein to sense a signal corresponding to the presence of the compound, the medical device is configured to sense light emanated from the indicator tag, wherein the light emanated from the indicator tag is in response to the indicator tag being exposed to light.

In an Example 3, the medical system of Example 2, wherein to sense light emanated from the indicator tag, the medical device is configured to sense a fluorescence of the light emanated by the indicator tag.

In an Example 4, the medical system of any of Examples 2 and 3, wherein to sense light emanated from the indicator tag, the medical device is configured to sense a fluorescence lifetime effect of the indicator tag.

In an Example 5, the medical system of any of Examples 2-4, wherein the processor is configured to determine the presence of the compound based on the received signal by determining at least one of: a ratio of an intensity of the emanated light to an intensity of the exposed light and a ratio of a wavelength of the emanated light to a wavelength of the exposed light.

In an Example 6, the medical system of any of Examples 2-5, wherein the exposed light comprises a first wavelength and a second wavelength and the emanated light comprises the first wavelength and the second wavelength; and wherein the processor is configured to determine the presence of the compound based on the received signal by determining: a first absorption of the first wavelength by the indicator tag, a second absorption of the second wavelength by the indicator tag and comparing the first absorption to the second absorption.

In an Example 7, the medical system of any of Examples 2-6, wherein the processor is configured to determine a concentration of the compound based on the received signal.

In an Example 8, the medical system of any of Examples 2-7, wherein the indicator tag comprises at least one of: a glucose-responsive fluorescent hydrogel and AcetonaPhthone phenyl ethyl Propionate Hydroxyl Tungstate.

In an Example 9, the medical system of any of Examples 1-8, wherein the compound is at least one of: hexane, ketones, catecholamine, cortisol, epinephrine and/or nor-epinephrine.

In an Example 10, the medical system of any of Examples 1-9, wherein the medical device is further configured to sense at least one of: the subject's acceleration, the subject's heart rate variability, a QT interval of the subject, a nerve transit time of the subject, the subject's reflex sensitivity, an autonomic tone of the subject and the subject's feedback to a cognitive test.

In an Example 11, a method comprises: sensing, using a medical device associated with a subject, a physiological parameter including at least one of: an acceleration, heart rate variability, a QT interval, a nerve transit time, reflex sensitivity, an autonomic and feedback to a cognitive test; correlating the sensed parameter to a blood glucose level; and determining, for the subject, using a processing device, an abnormal glycemic event based on the correlation of the sensed parameter to the blood glucose level.

In an Example 12, the method of Example 11, further comprising sensing a signal corresponding to a presence of a compound in at least one: an exhalation breath, interstitial fluid, blood and urine, wherein the compound is a surrogate for glucose.

In an Example 13, the method of Example 12, wherein the medical device is an implantable medical device and comprises an indicator tag exposed to interstitial fluid of the subject, wherein the indicator tag is responsive to the presence of the compound in interstitial fluid; wherein sensing a signal corresponding to a presence of a compound comprises exposing the indicator tag to light and receiving emanated light from the indicator tag in response to the indicator tag being exposed to light.

In an Example 14, the method of any of Examples 12-13, wherein the indicator tag comprises at least one of: a glucose-responsive fluorescent hydrogel and AcetonaPhthone phenyl ethyl Propionate Hydroxyl Tungstate.

In an Example 15, the method of any of Examples 12-14, wherein the compound is at least one of: hexane, ketones, catecholamine, cortisol, epinephrine and/or nor-epinephrine.

In an Example 16, a medical system comprises: a medical device associated with a subject, wherein the medical device is configured to sense a signal corresponding to a presence of a compound in at least one of: an exhalation breath, interstitial fluid, blood and urine, wherein the compound is a surrogate for glucose; and a processor communicatively coupled to the medical device, the processor configured to: receive the signal corresponding to the presence of the compound; determine the presence of the compound based on the received signal; and determine the subject is experiencing an abnormal glycemic event in response to the determined presence of the compound.

In an Example 17, the medical system of Example 16, wherein the medical device is an implantable medical device and comprises an indicator tag, wherein the indicator tag is responsive to the compound; and wherein to sense a signal corresponding to the presence of the compound, the medical device is configured to sense light emanated from the indicator tag, wherein the light emanated from the indicator tag is in response to the indicator tag being exposed to light.

In an Example 18, the medical system of Example 17, wherein to sense light emanated from the indicator tag, the medical device is configured to sense a fluorescence of the light emitted by the indicator tag.

In an Example 19, the medical system of Example 17, wherein to sense light emanated from the indicator tag, the medical device is configured to sense a fluorescence lifetime effect of the indicator tag.

In an Example 20, the medical system of Example 17, wherein the processor is configured to determine at least one of: a ratio of an intensity of the emanated light to an intensity of the exposed light and a ratio of a wavelength of the emanated light to a wavelength of the exposed light.

In an Example 21, the medical system of Example 17, wherein the exposed light comprises a first wavelength and a second wavelength and the emanated light comprises the first wavelength and the second wavelength; and wherein the processor is configured to determine the presence of the compound based on the received signal by determining: a first absorption of the first wavelength by the indicator tag, a second absorption of the second wavelength by the indicator tag and comparing the first absorption to the second absorption.

In an Example 22, the medical system of Example 17, wherein the processor is further configured to determine a concentration of the compound based on the received signal.

In an Example 23, the medical system of Example 17, wherein the indicator tag comprises at least one of: a glucose-responsive fluorescent hydrogel and AcetonaPhthone phenyl ethyl Propionate Hydroxyl Tungstate.

In an Example 24, the medical system of Example 16, wherein the compound is at least one of: hexane, ketones, catecholamine, cortisol, epinephrine and/or nor-epinephrine.

In an Example 25, the medical system of Example 16, wherein the medical device is further configured to sense at least one of: the subject's acceleration, the subject's heart rate variability, a QT interval of the subject, a nerve transit time of the subject, the subject's reflex sensitivity, an autonomic tone of the subject and the subject's feedback to a cognitive test.

In an Example 26, a method comprises: sensing, using a medical device associated with a subject, a physiological parameter including at least one of: an acceleration, heart rate variability, a QT interval, a nerve transit time, reflex sensitivity, an autonomic and feedback to a cognitive test; correlating the sensed parameter to a blood glucose level; and determining, for the subject, using a processing device, an abnormal glycemic event based on the correlation of the sensed parameter to the blood glucose level.

In an Example 27, the method of Example 26, further comprising sensing a signal corresponding to a presence of a compound in at least one: an exhalation breath, interstitial fluid, blood and urine, wherein the compound is a surrogate for glucose.

In an Example 28, the method of Example 27, wherein the medical device is an implantable medical device and comprises an indicator tag exposed to interstitial fluid of the subject, wherein the indicator tag is responsive to the presence of the compound in interstitial fluid; wherein sensing a signal corresponding to a presence of a compound comprises exposing the indicator tag to light and receiving emanated light from the indicator tag in response to the indicator tag being exposed to light.

In an Example 29, the method of Example 28, wherein sensing a signal corresponding to a presence of a compound comprises sensing a fluorescence of the light emanated by the indicator tag.

In an Example 30, the method of Example 28, wherein sensing a signal corresponding to a presence of a compound comprises sensing a fluorescence lifetime effect of the indicator tag.

In an Example 31, the method of Example 28, wherein correlating the sensed parameter to a blood glucose level comprises determining at least one of: a ratio of an intensity of the emanated light to an intensity of the exposed light and a ratio of a wavelength of the emanated light to a wavelength of the exposed light.

In an Example 32, the method of Example 31, wherein the exposed light comprises a first wavelength and a second wavelength and the emanated light comprises the first wavelength and the second wavelength; and wherein determining a ratio of the emanated light to the exposed light comprises determining: a first absorption of the first wavelength by the indicator tag, a second absorption of the second wavelength by the indicator tag and comparing the first absorption to the second absorption.

In an Example 33, the method of Example 28, further comprising determining a concentration of the compound based on the received emanated light.

In an Example 34, the method of Example 27 wherein the indicator tag comprises at least one of: a glucose-responsive fluorescent hydrogel and AcetonaPhthone phenyl ethyl Propionate Hydroxyl Tungstate.

In an Example 35, the method of Example 27, wherein the compound is at least one of: hexane, ketones, catecholamine, cortisol, epinephrine and/or nor-epinephrine.

While multiple embodiments are disclosed, still other embodiments of the present disclosure will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic illustration of a system for determining an abnormal glycemic event using one or more surrogate for glucose, in accordance with embodiments of the present disclosure.

FIG. 2 is a block diagram depicting an illustrative medical device for determining an abnormal glycemic event using one or more surrogate for glucose, in accordance with embodiments of the present disclosure.

FIGS. 3A-3D are images of an illustrative indicator tag depicting different fluorescence responses to different surrogate compounds, in accordance with embodiments of the present disclosure.

FIG. 4 depicts a graph of a fluorescence intensity change emanated from an indicator tag in response to different concentrations of a surrogate compound, in accordance with embodiments of the present disclosure.

FIG. 5 is a graph depicting the varying light absorption of an acetone treated sample and an ethanol treated sample as a function of wavelength, in accordance with embodiments of the present disclosure.

FIG. 6 is a graph depicting the delay in the varying light absorption of an acetone treated sample as a function of wavelength, in accordance with embodiments of the present disclosure.

FIG. 7 is a flow diagram depicting an illustrative process for determining an abnormal glycemic event using one or more surrogates for glucose, in accordance with embodiments of the present disclosure.

While the disclosed subject matter is amenable to various modifications and alternative forms, specific embodiments have been shown by way of example in the drawings and are described in detail below. The intention, however, is not to limit the disclosed subject matter to the particular embodiments described. On the contrary, the disclosed subject matter is intended to cover all modifications, equivalents, and alternatives falling within the scope of the disclosed subject matter as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

The present disclosure relates to systems and methods for determining an abnormal glycemic event. To do so, in embodiments, the systems and methods disclosed herein may measure one or more physiological parameters that are surrogates for blood glucose concentration (hereinafter referred to as “surrogates”) to determine when a subject is experiencing an abnormal glycemic event. “Surrogates,” as used herein, are indicators, other than glucose, of physiological responses to glucose imbalance. In embodiments, the surrogates may indicate physiological responses to only abnormal glucose levels. Alternatively, in embodiments, the surrogates may indicate physiological responses to glucose levels for all glucose ranges, including abnormal ranges of glucose and normal ranges of glucose. Surrogates that are compounds may be referred to herein as “surrogate compounds” and surrogates that are other physiological parameters may be referred to herein as “surrogate parameters.”

Generally, blood-glucose levels should be between approximately between 4.2 millimoles per liter (mmol/L) to 5.0 mmol/L when a subject is fasted and may rise as high as 7.0 mmol/L after a subject has eaten. Accordingly, an abnormal glycemic event, as used herein, may be a blood-glucose concentration that is not between 4.2 mmol/L and 5.0 mmol/L when the subject is in a fasted state and greater than 7.0 mmol/L after a meal. When the subject's glucose is exceeds these limits, the subject is said to be experiencing a hyperglycemic event and when the subject's glucose levels are below 4.2 mmol/L, the subject is said to be experiencing a hypoglycemic event.

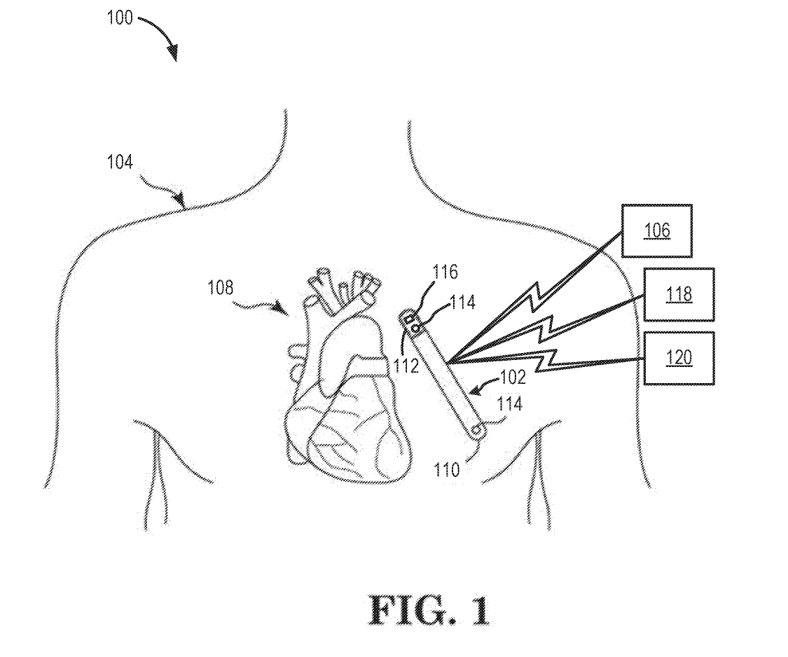

FIG. 1 is a schematic illustration of a system 100 including a medical device (MD) 102 associated with a subject's body 104 and configured to be communicatively coupled to a processor 106. The system 100 may be used to monitor (e.g., determine, sense and/or record) one or more parameters of the subject's body 104 in order to diagnose and/or provide therapy to the subject, in accordance with embodiments of the disclosure. In embodiments, the MD 100 may be located external to the subject's body 104 where the MD 102 may be configured to monitor physiological parameters of the subject. Alternatively, in embodiments, the MD 100 may be implanted subcutaneously within an implantation location or pocket, for example, in the subject's chest, abdomen, head, leg and/or arm, where the MD 102 may be configured to monitor one or more physiological parameters of the of the subject. For example, the MD 102 may be located in a subject's interstitial fluid, serous fluid, gastric fluid, blood, urine, an organ, breath and/or the like.

Additionally or alternatively, in embodiments, the MD 102 may be configured to monitor other physiological parameters associated with the subject's circulatory system 108. For example, the MD 102 may be an implantable cardiac monitor (ICM) (e.g., an implantable diagnostic monitor (IDM), an implantable loop recorder (ILR), etc.) configured to monitor physiological parameters such as, for example, the subject's cardiac activation signals, heart sounds, pulsations of arteries, oxygen saturations, and/or the like.

Additionally or alternatively, in embodiments, the MD 102 may also be configured to monitor other physiological parameters associated with the subject's respiratory system. For example, the MD 102 may be an implantable respiratory monitor (IRM) configured to monitor physiological parameters such as, for example, the subject's respiratory rate, tidal volume, respiratory pattern, airflow, oxygen saturations, and/or the like. However, these are only examples and not meant to be limiting.

Additionally or alternatively, in embodiments, the MD 102 may be configured to monitor physiological parameters that may include one or more signals indicative of a subject's physical activity level and/or metabolic level, such as an acceleration signal. In embodiments, the MD 102 may be configured to monitor physiological parameters associated with one or more other organs, systems, and/or the like. For example, the MD 102 may include sensors or circuitry for detecting cardiac system signals, circulatory system signals, nervous system signals, respiratory system signals, and/or signals related to subject activity.

Additionally or alternatively, in embodiments, the MD 102 may be configured to sense intrathoracic impedance, from which various respiratory parameters may be derived, including, for example, respiratory tidal volume and minute ventilation. In embodiments, the MD 102 may be configured to sense cardiac impedance, from which various cardiac parameters may be derived, including, for example, left and right ventricular activity. Sensors and associated circuitry may be incorporated in connection with the MD 102 for detecting one or more body movements, body postures and/or position related signals. For example, accelerometers and/or GPS devices may be employed to detect tremors, shaking, imbalance patterns, subject activity, subject location, body orientation, and/or torso position. In embodiments, one or more body movements, body postures and/or position related signals may be used as a secondary and/or confirmatory signal to other signals, for example, the signal indicative of a surrogate compound and/or surrogate compound concentration.

Additionally or alternatively, in embodiments, the MD 102 may be configured to monitor other physiological parameters associated with the subject's muscular system, skeletal system, nervous system, lymphatic system, respiratory system and/or endocrine system. For example, the MD 102 may include sensor or circuitry for detecting nerve transit time, reflex sensitivity, autonomic tones and/or cognitive feedback to one or more cognitive tests.

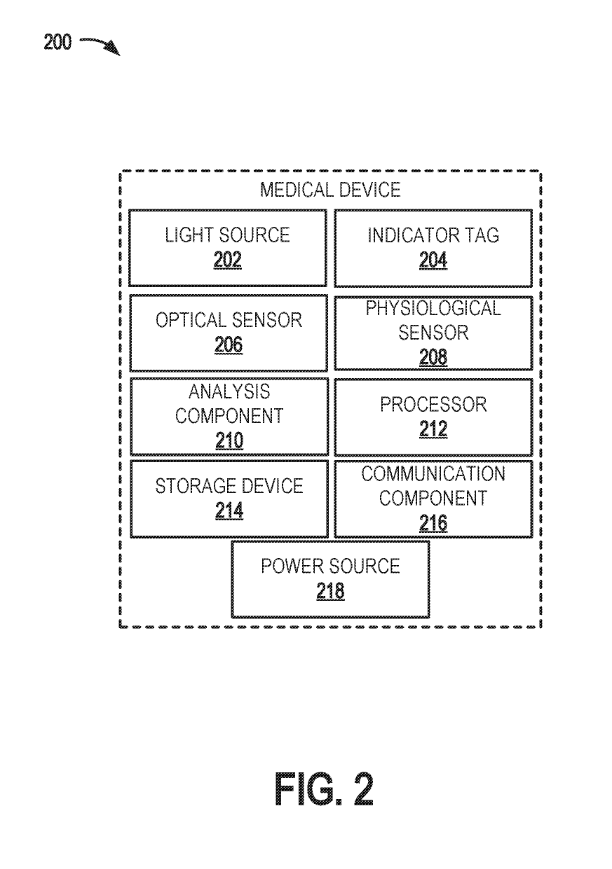

In embodiments, the processing device 106 may determine when the subject is experiencing an abnormal glycemic event based on one or more of the above physiological parameters, i.e., surrogate parameters, as explained in more detail in relation to FIG. 2 below.

For purposes of illustration, and not of limitation, various embodiments of devices that may be used to monitor physiological parameters in accordance with the present disclosure are described herein in the context of MDs that may be implanted under the skin in the chest region of a subject. In embodiments, however, the MD 102 may include any type of MD, any number of different components of a MD, and/or the like having a housing and being configured to be associated with a subject's body 104.

For example, the MD 102 may include a control device, a monitoring device, a pacemaker, an implantable cardioverter defibrillator (ICD), a subcutaneous implantable cardioverter defibrillator (S-ICD), a leadless implantable cardioverter defibrillator (L-ICD), a cardiac resynchronization therapy (CRT) device, a neural stimulation device, and/or the like, and may be an implantable medical device known in the art or later developed, for providing therapy and/or diagnostic data about the subject's body and/or the MD 102. In various embodiments, the MD 102 may include both defibrillation and pacing/CRT capabilities (e.g., a CRT-D device).

The MD 102 may be configured to monitor at regular intervals, continuously, and/or in response to a detected event. In embodiments, such a detected event may be detected by one or more sensors of the MD 102, another MD (not shown), an external device (not shown), and/or the like. In addition, the MD 102 may be configured to detect a variety of parameters and/or concentrations thereof that may be used in connection with various diagnostic, therapeutic, and/or monitoring implementations.

As shown, the MD 102 may include a housing 110 having a header 112 that is arranged near an end of the MD 102. The housing 110 may include any number of different shapes, sizes, and/or features. In embodiments, the MD 102 may include any number of electrodes 114 and/or other types of sensors such as, e.g., sound sensors, pressure sensors, impedance sensors, optical sensors, thermometers, barometers, motion or impact sensors (e.g., accelerometers, inertial measuring units (IMUs)), and/or the like) in any number of various types of configurations. In embodiments, the processing device 106 may determine when the subject is experiencing an abnormal glycemic event based on one or more outputs of the electrodes 114 and/or other types of sensors, as explained in more detail in relation to FIG. 2 below.

In embodiments, the MD 102 may include an indicator tag 116 that is responsive to one or more surrogate compounds and/or one or more surrogate compound concentrations. Examples of surrogate compounds include, but are not limited to: hexane, ketones (e.g., beta-hydroxybutyrate, acetone, acetoacetic acid, glucagon, epinephrine, cortisol) and/or the like.

For purposes of illustration, and not of limitation, various embodiments for measuring a surrogate compound are described in relation to the indicator tag 116 (or the indicator tag 204 depicted in FIG. 2 below). In embodiments, however, the MD 102 (or the MD 200 depicted in FIG. 2) may include, in addition to an indicator tag 116 or alternatively to an indicator tag 116, a sensor that is configured to measure a surrogate compound in a subject's interstitial fluid, serous fluid, gastric fluid, blood, urine, an organ, breath and/or the like.

In embodiments, the indicator tag 116 is in communication with the portion of the subject in which the surrogate compound may be present. For example, the indicator tag 116 may be in communication with the subject's interstitial fluid, serous fluid, gastric fluid, blood, urine, an organ, breath and/or the like that may include the surrogate compound. To do so, the indicator tag 116 may be located on a portion of the MD 102 such that the indicator tag 116 is in communication the subject's interstitial fluid, serous fluid, gastric fluid, blood, urine, an organ, breath and/or the like that may include the surrogate compound. For example, in embodiments, the indicator tag 116 may be located on a portion of the MD 102 that is directly exposed to the subject's interstitial fluid, serous fluid, gastric fluid, blood, urine, an organ, breath and/or the like that may include the surrogate compound. As another example, the indicator tag 116 may be located on a portion of the MD 102 and covered by a layer of material that is permeable to the surrogate compound. In embodiments, the indicator tag 116 may be covered by a layer of material to prolong the useful life of the indicator tag 116.

In embodiments, the indicator tag 116 may include one or more indicator tags 116. In embodiments where more than one indicator tag 116 is included, one indicator tag 116 may be used as a redundancy check for the other indicator tag 116. Additionally or alternatively, each indicator tag 116 may be responsive to the same surrogate compound and/or surrogate compound concentration; or, each indicator tag 116 may be responsive to different surrogate compounds and/or different surrogate compound concentrations. Additionally or alternatively, in embodiments, one indicator tag 116 may be covered by a surface that decays over time so that a first indicator tag 116 will be used for a first period of time and a second indicator tag 116 that is covered by a surface that decays over time will be used for a second period of time, after the first period of time. While two indicator tags 116 are discussed, there may be three, four, five, six, etc. indicator tags 116. Including indicator tags 116 that are used during different time periods may prolong the useful life of the MD 102.

To determine a response of the indicator tag 116, that is indicative of a surrogate compounds and/or a surrogate compound concentration, the indicator tag 116 may be exposed to light emitted from a light source 118. In response to being exposed to light, the indicator tag 116 may emanate light that is indicative of a surrogate compounds and/or a surrogate compound concentration contacting the indicator tag 116. In embodiments, the light emanated from the indicator tag 116 may be re-radiated light via fluorescence. In embodiments, the light emanated from the indicator tag 116 may be light that is reflected by the indicator tag 116. Examples of properties of the indicator tag 116 that may change in response to different surrogate compounds and/or surrogate compound concentrations include, but are not limited to, the type and/or amount of light that the indicator tag 116 absorbs and reflects, the fluorescence of the indicator tag 116 and/or the fluorescence lifetime effect. These examples are explained in more detail below in relation to FIG. 2.

The emanated light from the indicator tag 116 may be sensed using an optical sensor 120. The sensed light by the optical sensor 120 may be stored in memory and/or communicated to the processor 106 via one or more signals. In embodiments, the processor 106 may be configured to determine a surrogate compound and/or a surrogate compound concentration based on the received signal from the optical sensor 120. Based on the determined surrogate compounds and/or a surrogate compound concentration, the processing device 106 may determine when the subject is experiencing an abnormal glycemic event, as explained in more detail in relation to FIG. 2 below.

Additionally or alternatively, the processor 106 may be configured to transition from a lower-power state to a higher-power state in response to receiving a sensed light pulse from the optical sensor 120. After converting to a higher-power state, the processor 106 may be configured to determine an abnormal glycemic event. Additionally or alternatively, the processor 106 may be configured to transition from a lower-power state to a higher-power state after receiving a signal via a non-wireless and/or wireless communication link. In embodiments, the processor 106 may transition from a higher-power state to a lower-power state after determining an abnormal glycemic event.

In embodiments, the processor 106, the light source 118 and/or the optical sensor 120 may be incorporated into the MD 102 or external to the MD 102. For example, in embodiments where the processor 106, the light source 118 and/or the optical sensor 120 are external to the MD 102, the processor 106, the light source 118 and/or the optical sensor 120 may be incorporated into another MD (not shown). Alternatively, in embodiments where the processor 106, the light source 118 and/or the optical sensor 120 are external to the MD 102, the processor 106, the light source 118 and/or the optical sensor 120 may be positioned on the subject, near the subject, or in any location external to the subject.

In embodiments, the MD 102 and the processor 106 may communicate via a non-wireless and/or wireless communication link. For example, the MD 102 and the processor 106 may be communicatively coupled via a bus. As another example, the MD 102 and the processor 106 may be communicatively coupled through a short-range radio link, such as Bluetooth, IEEE 802.11, and/or a proprietary wireless protocol. The term “communication link” may refer to an ability to communicate some type of information in at least one direction between at least two devices, and should not be understood to be limited to a direct, persistent, or otherwise limited communication channel. That is, according to embodiments, the communication link may be a persistent communication link, an intermittent communication link, an ad-hoc communication link, and/or the like. The communications link may facilitate uni-directional and/or bi-directional communication between the MD 102 and the processor 106. Data and/or control signals may be transmitted between the MD 102 and the processor 106 to coordinate the functions of the MD 102 and/or the processor 106. In embodiments, subject data may be downloaded from one or more of the MD 102 and the processor 106 periodically or on command. The physician and/or the subject may communicate with the MD 102 and the processor 106, for example, to determine an abnormal glycemic event and/or to initiate, terminate, or modify the determination of an abnormal glycemic event and/or to administer therapy.

The illustrative system 100 shown in FIG. 1 is not intended to suggest any limitation as to the scope of use or functionality of embodiments of the subject matter disclosed throughout this disclosure. Neither should the illustrative system 100 be interpreted as having any dependency or requirement related to any single component or combination of components illustrated in FIG. 1. For example, in embodiments, the illustrative system 100 may include additional components. Additionally, any one or more of the components depicted in FIG. 1 can be, in embodiments, integrated with various ones of the other components depicted therein (and/or components not illustrated). Any number of other components or combinations of components can be integrated with the illustrative system 100 depicted in FIG. 1, all of which are considered to be within the ambit of this disclosure.

FIG. 2 is a block diagram depicting an illustrative medical device 200 for determining an abnormal glycemic event using one or more surrogates for glucose, in accordance with embodiments of the present disclosure. The MD 200 may be, be similar to, include, or be included in, the MD 102 depicted in FIG. 1. For example, the MD 200 may be external to a subject, implanted in a subject's chest, abdomen head, leg and/or arm, and/or may be implanted in interstitial fluid, serous fluid, gastric fluid, blood, urine, an organ, breath and/or the like. Embodiments of the medical 200 may include more than one MD 200.

According to embodiments illustrated in FIG. 2, the MD 200 includes a light source 202, an indicator tag 204, an optical sensor 206, a physiological sensor 208, an analysis component 210, a processor 212, a storage device 214, a communication component 216 and/or a power source 218.

In embodiments, the light source 202 may be, be similar to, include, or be included in, the light source 118 depicted in FIG. 1. The light source 202 is configured to emit light. In embodiments, the light emitted from the light source 202 may be continuous light, a light pulse and/or a series of more than one light pulse (e.g., two light pulses, three light pulses, etc.). In embodiments where a series of light pulses are emitted from the light source 202, the light pulses may be of different durations and/or intensities. In embodiments, a first light pulse of a series of light pulses may be used to transition the MD 200 from a lower-power state to a higher-power state, as described below.

Additionally or alternatively, the light emitted from the light source 202 may be comprised of a single narrowband of wavelengths or more than one narrowband of wavelengths. In embodiments, the light emitted from the light source 202 may be two narrowband sources, three narrowband source, etc. Additionally or alternatively, in embodiments, more than one narrowband of wavelengths may be produced using an LED and a specific phosphor for the type of narrowband wavelength that is to be obtained. For example, white light may be produced using a blue or ultraviolet light-emitting diode (LED) and a phosphor coating. The blue or ultraviolet photons generated by the blue or ultraviolet LED either travel through the phosphor layer unaltered, or they are converted into yellow photons in the phosphor layer. Some of the yellow photons may combine with the blue or ultraviolet photons to generate white light. A light source 202 that emits more than one narrowband of wavelengths may be used to reduce the likelihood that changes sensed by the optical sensor 206 are determined to be changes in a surrogate compound and/or surrogate concentration when instead the changes are due to either output changes of the light source 202 and/or path loss changes, as explained below.

The light emitted from the light source 202 is directed at the indicator tag 204. The light emitted from the light source 202 and directed at the indicator tag 204 may include more than one narrowband of wavelengths. The indicator tag 204 is exposed to at least a portion of the emitted light that is directed at the indicator tag 204. In response to being exposed to some or all of the emitted light from the light source 202, the indicator tag 204 is configured to emanate light. In embodiments, the light emanated from the indicator tag 204 may include more than one narrowband of wavelengths. In embodiments, the type and/or amount of light that the indicator tag 204 emanates may be responsive to the environment of the indicator tag 204. That is, as described above, the indicator tag 204 may vary the type and/or amount of light that it emanates in response to a surrogate compound and/or a surrogate compound concentration. In embodiments, the light emanated from the indicator tag 204 may be re-radiated light via fluorescence. In embodiments, the light emanated from the indicator tag 204 may be light that is reflected by the indicator tag 204.

In embodiments, the amount of light that is emanated by the indicator tag 204 may be responsive to the different wavelengths of light to which the indicator tag 204 is exposed, surrogate compounds to which the indicator tag 204 is exposed and/or surrogate compound concentrations to which the indicator tag 204 is exposed. For example, in response to being exposed to a first surrogate compound, the indicator tag 204 may absorb more green light than red light and, therefore, may emanate more red light than green light. That is, the intensity of the green light that is emanated by the indicator tag 204 is greater than the intensity of the red light that is emanated. However, in response to being exposed to a non-surrogate compound, the indicator tag 204 may absorb more red light than green light and, therefore, may emanate more green light than red light.

As another example, the fluorescence of the indicator tag 204 may change in response to one or more different surrogate compounds and/or different surrogate compound concentrations. For example, in response to being exposed to a first surrogate compound, the indicator tag 204 may have a first fluorescence color. Further, in response to being exposed to a non-surrogate compound, the indicator tag 204 may have a second fluorescence color.

As even another example, the fluorescent lifetime effect of the indicator tag 204 may change in response to one or more different surrogate compounds and/or different surrogate compound concentrations. That is, the emanated light by the indicator tag 204 may be delayed by different times that correspond to different surrogate compounds and/or different concentrations of surrogate compounds. For example, in response to being exposed to a first surrogate compound, the emanated light from the indicator tag 204 may be delayed by a first time. Further, in response to being exposed to a second surrogate compound, the indicator tag 204 may be delayed by a second time.

In embodiments, the indicator tag 204 may be comprised of, but not limited to, for example: a glucose-responsive fluorescent hydrogel, AcetonaPhthone phenyl ethyl Propionate Hydroxyl Tungstate (APPHT), etc. Surrogate compounds that the indicator tag 204 may be responsive to include, but are not limited to, for example, hexane, ketones, catecholamine, cortisol, epinephrine and/or nor-epinephrine.

The indicator tag 204 may be adhered and/or bonded to the MD 200 using one or more adhesives and/or bonding techniques for MDs 200. Alternatively, in embodiments, the indicator tag 204 may be configured to adhere to the MD 200 without the use of an adhesive. In embodiments, the indicator tag 204 may be located on a portion of the MD 200 that is directly exposed to a surrogate compound. In embodiments, the indicator tag 204 may be covered by a layer that is permeable to the surrogate compound. In embodiments, the indicator tag 204 may be covered by a layer to prolong the useful life of the indicator tag 204.

While only one indicator tag 204 is depicted in FIG. 2, in embodiments, more than one indicator tag 204 may be disposed on the outer surface of the MD 200. In embodiments where more than one indicator tag 204 is included, one indicator tag 204 may be used as a redundancy check for the other indicator tag 204. Additionally or alternatively, each indicator tag 204 may be responsive to the same surrogate compound and/or surrogate compound concentration; or, each indicator tag 204 may be responsive to different surrogate compounds and/or different surrogate compound concentrations. Additionally or alternatively, in embodiments, one indicator tag 204 may be covered by a surface that decays over time so that a first indicator tag 204 will be used for a first period of time and a second indicator tag 204 that is covered by a surface that decays over time will be used for a second period of time, after the first period of time. While two indicator tags 204 are discussed, there may be three, four, five, six, etc. indicator tags 204. Including indicator tags 204 that are used during different time periods may prolong the useful life of the MD 200.

In embodiments, the indicator tag 204 may include one or more filters (e.g., a bandpass filter) and/or be coupled to one or more filters for filtering out one or more wavelengths of light.

Additionally or alternatively, in embodiments, one or more waveguides may couple emitted light from the light source 202 to the indicator tag 204. In embodiments where the light source 202 emits more than one narrowband of wavelengths, a single waveguide may couple the emitted light from the light source 202 to the indicator tag 204. Alternatively a respective waveguide for each narrowband of wavelengths of light may couple the emitted light from the light source 202 to the indicator tag 204. In embodiments, the one or more waveguides may include a filter for filtering out one or more wavelengths of light.

In embodiments, any number of mitigation systems and methods may be used to increase useful life of the indicator tag 204 and/or the MD 200. For example, the indicator tag 204 may be coated with a thin-film membrane. Other examples of mitigation systems and methods that may be used to increase the useful life of the indicator tag 204 and/or the MD 200 are discussed in U.S. patent application Ser. No. 14/822,779, entitled “Implantable Medical Device Coating for Wetting and Microbial Resistance,” filed on Aug. 10, 2015; U.S. patent application Ser. No. 14/255,738, entitled “Medical Implant Having a Conductive Coating,” filed on Apr. 17, 2014; U.S. patent application Ser. No. 13/680,590, entitled “Fibrous Matrix Coating Materials,” filed on Nov. 19, 2012; and/or U.S. Pat. No. 9,364,662, entitled “Implantable Lead Having a Lumen with a Wear-Resistant Liner, the disclosures of which are hereby expressly incorporated herein by reference.

The optical sensor 206 is configured to sense at least a portion of the emanated light from the indicator tag 204. In embodiments, the optical sensor 206 may be configured to sense the intensity of the emanated light and/or the color of the emanated light. The optical sensor 206 may be, for example, a photodetector. In embodiments, the optical sensor 206 may include a single sensor configured to sense a single narrowband of wavelengths. Alternatively, in embodiments, the optical sensor 206 may include a plurality of sensors, such that each sensor senses a respective narrowband of wavelengths. In embodiments, the optical sensor 206 may include one or more filters that filter out one or more wavelengths of light, so that only a specific narrowband of wavelength is sensed by the optical sensor 206.

In embodiments, one or more waveguides may couple the emanated light from the indicator tag 204 to the optical sensor 206. In embodiments where the emanated light includes more than one narrowband of wavelengths of light, a single waveguide may couple the emanated light from the indicator tag 204 to the optical sensor 206. Alternatively, a respective waveguide for each narrowband of wavelengths may couple the emanated light from the indicator tag 204 to the optical sensor 206. In embodiments, the one or more waveguides may be configured for dual directionality. That is, a waveguide may couple energy from the light source 202 to the indicator tag 204 and from the indicator tag 204 to the optical sensor 206. In embodiments, the one or more waveguides may include one or more filters for filtering out one or more wavelengths of light.

After the optical sensor 206 senses at least a portion of the emanated light from the indicator tag 204, one or more signals corresponding to the sensed emanated light and/or corresponding to the sensed physiological parameters may be sent to and received by the analysis component 210. From the received signals, the analysis component 210 may determine an abnormal glycemic event.

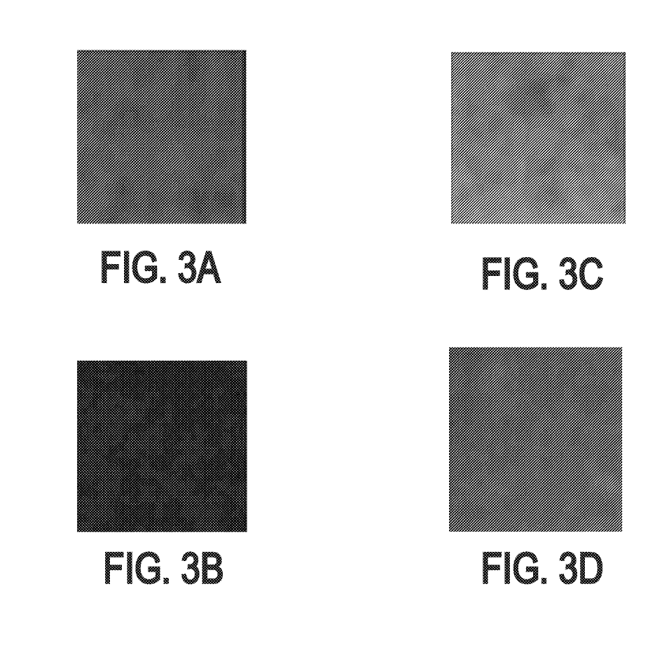

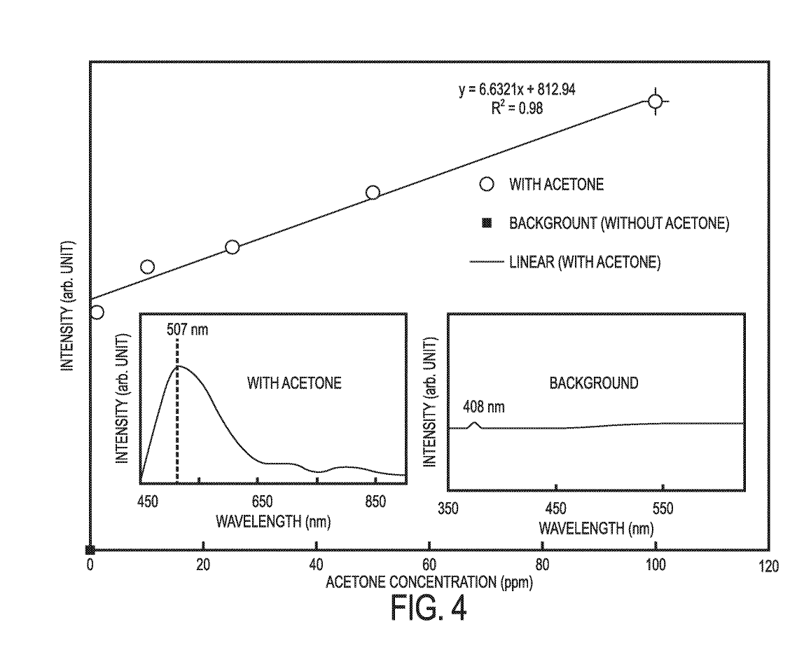

As stated above, in response to different surrogate compounds and/or different surrogate compound concentrations, the indicator tag 204 may change the amount of light that emanates from the indicator tag 204. For example, the amount of light the indicator tag 204 reflects, the fluorescence of the indicator tag 204 and/or the fluorescence lifetime effect of the indicator tag 204 may change in response to different surrogate compounds and/or different surrogate compound concentrations. For example, in response to being exposed to a surrogate compound, the indicator tag 204 may change its fluorescence. When the optical sensor 206 senses a fluorescence of the indicator tag 204, a signal may be sent to the analysis component 210. The analysis component may determine the fluorescence that was sensed by the optical sensor 206. After which, the analysis component 210 may correlate the determined fluorescence to one or more surrogate compounds, one or more surrogate compound concentrations and/or one or more non-surrogate compounds. Images of an illustrative indicator tag (e.g., the indicator tag 204) depicting different fluorescence responses to different surrogate compounds is provided in FIGS. 3A-3D below. A graph depicting a fluorescence intensity change emanated from an indicator tag (e.g., the indicator tag 204) in response to different concentrations of a surrogate compound is illustrated in FIG. 4 below.

As another example, the analysis component 210 may determine the presence of a surrogate compound and/or a surrogate compound concentration based on the fluorescence lifetime effect of the indicator tag 204. That is, the analysis component 210 may determine a delay in the emanated light by the indicator tag 204 after the indicator tag 204 is stimulated by light emitted from the light source 202. After which, the analysis component may correlate the delay to the presence of a specific surrogate compound and/or a surrogate compound concentration to which the indicator tag 204 responds. Similarly, in embodiments, the light emitted from the light source 202 may include more than one wavelength and the plurality of wavelengths of light may be analyzed by the analysis component 210 to determine whether the changes in emanated light of the indicator tag 204 are due to the presence of a surrogate compound, a surrogate compound concentration, changes in the output intensity of the light source 202 and/or changes in path loss changes, as described above.

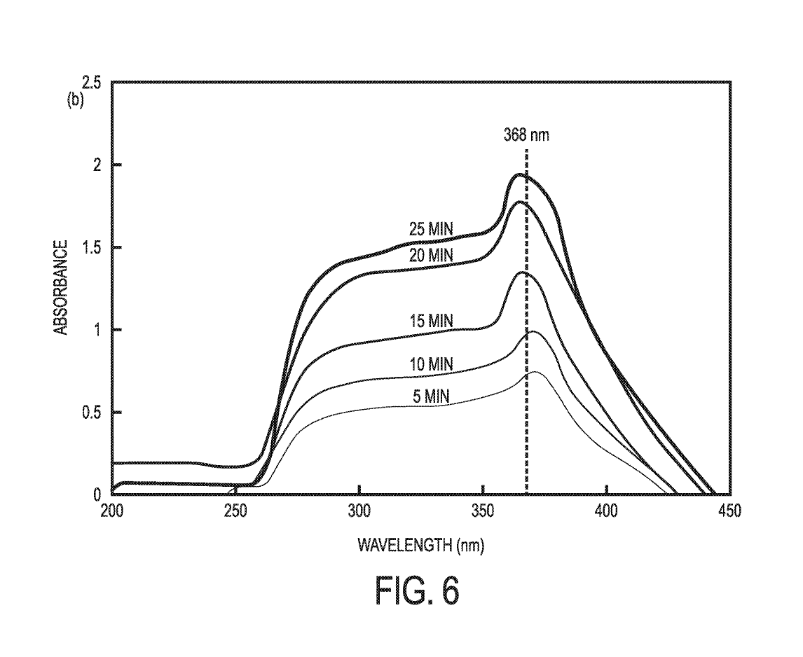

As another example, in embodiments where the indicator tag 204 changes the amount of light that it absorbs and/or emanates in response to a surrogate compound and/or surrogate compound concentration, the analysis component 210 may determine a ratio between the intensity and/or the wavelength of the received light by the optical sensor 206, from the indicator tag 204, and the intensity and/or the wavelength of the emitted light from the light source 202. The analysis component 210 may then correlate the ratio to a specific surrogate compound and/or specific surrogate compound concentration to which the indicator tag 204 responds. A graph depicting an absorbance change of an indicator tag 206 in response to a surrogate compound and a non-surrogate compound is depicted in FIG. 6 below.

In embodiments, the analysis component 210 may determine the ratio of received light to emitted light for more than one narrowband of wavelengths and compare the ratios for the different narrowbands of wavelengths. By determining ratios of received light to emitted light for multiple narrowbands of wavelengths and comparing the ratios to one another, the analysis component 210 may determine whether the output of the light source 202 has changed and/or whether there are any path loss changes.

For example, assume the absorption of a first wavelength by the indicator tag 204 decreases when a surrogate compound is present and/or when the surrogate compound concentration increases and the absorption of a second wavelength stays relatively constant when the surrogate compound is present and/or for different concentrations of the surrogate compound. Further assume the light emitted from the light source 202 is not measured each time light is emitted, but instead is assumed to be constant. Finally, assume that the intensity of the emitted light has decreased and/or the path loss of the light has increased. As such, if only the ratio of received light to emitted light for the first wavelength, which is dependent on the presence of the surrogate compound and/or the surrogate compound concentration, was determined, the ratio would be skewed down because less light would be received due to the decreasing intensity of the emitted light and/or due to the increasing path loss for the light. However, the analysis component 210 may be unable to determine whether the decrease in the ratio was due to the presence of the surrogate compound, an increasing concentration of the surrogate compound, a change in intensity of emitted light and/or a path loss change. On the contrary, if two different ratios were computed for the two different wavelengths, the analysis component 210 may determine whether the intensity of the emitted light has decreased and/or the path loss of the light has increased. That is, the ratio of the received light to the emitted light for the second wavelength would be skewed down. However, the ratio of the received light to the emitted light should be constant for the second wavelength because the second wavelength is independent of the presence of the surrogate compound and/or the surrogate compound concentration. Accordingly, the analysis component 210 could correct the ratio of the received light to the emitted light for the first wavelength based on the skewed ratio for the second wavelength. The analysis component 210 can, therefore, determine the presence of a surrogate compound and/or a surrogate compound concentration based on the corrected ratio for the first wavelength.

The physiological sensor 208 is configured to sense one or more surrogate parameters of a subject. For example, the physiological sensor 208 may be configured to sense, one or more signals indicative of a tremor, shaking, imbalance patterns, subject location, body orientation, torso position, subject physical activity level and/or metabolic level (e.g., an acceleration signal), one or more signals associated with the subject's circulatory system (e.g., heart rate, heart rate variability, a QT interval), one or more signals associated with autonomic tones, nerve transit time and/or reflex sensitivity. As another example, the physiological sensor 208 may sense signals corresponding to cognitive feedback (e.g., response given, response time, amount of questions answered and/or the like) to one or more cognitive tests. After the physiological sensor 208 senses one or more surrogate parameters, one or more signals corresponding to the sensed surrogate parameters may be sent to and received by the analysis component 210. From the received signals, the analysis component 210 may determine an abnormal glycemic event.

As an example, the physiological sensor 208 may be configured to sense an adrenaline response, which may be correlated to a subject having low blood sugar. That is, when a subject experiences low blood sugar (e.g., less than 4.2 mmol/L), the subject may twitch, shake, experience a high-frequency wobble and/or sweat. The twitching, shaking, wobbling and/or sweating may be sensed by the physiological sensor 208. For example, the physiological sensor 208 may be an accelerometer to measure movement, a moisture sensor, a temperature sensor to measure skin temperature and/or a chemical sensor to measure one or more metabolites and/or electrolytes on the skin. One or more signals indicative of a twitch, a shake, a high-frequency wobble and/or sweating may be sent to the analysis component 210. The analysis component 210 may determine whether the subject is experiencing an adrenaline response based on the received signals. In embodiments, the analysis component 210 may compare to signals indicative of the twitch, the shake, the high frequency wobble and/or the sweating to a baseline for the subject. If the twitch, shake, high frequency wobble and/or sweating varies by more than a threshold (e.g., 10%, 20%, 30% and/or the like) from a baseline twitch, shake, wobble and/or sweat, then the analysis component 210 may determine the subject to have low blood sugar. In embodiments, the analysis component 210 may be configured to determine a baseline of the subject when the subject is not experiencing low or high blood sugar.

As another example, the physiological sensor 208 may be configured to sense a central nervous system (CNS) response, which may be correlated to having low blood sugar, since the CNS requires glucose but not insulin. For example, the physiological sensor 208 may sense heart-rate variability, a QT interval, nerve transit time, reflex sensitivity and/or an autonomic tone. Regarding heart-rate variability, one or more signals indicative of sensed heart rate by the physiological sensor 208 may be sent to the analysis component 210. The analysis component 210 may determine whether the subject's heart rate has changed from a lower heart rate to a higher rate faster than a normal heart rate cyclic variation. If the heart rate variability is not within a threshold (e.g., 10%, 20%, 30% and/or the like) of the normal heart rate cyclic variation for the subject for the subject, then the analysis component 210 may determine the subject to have low blood sugar. Similar to above, in embodiments, the analysis component 210 may be configured to determine a normal heart rate cyclic variation of the subject when the subject is not experiencing low or high blood sugar.

Regarding a QT interval for the subject, one or more signals indicative of a sensed QT interval by the physiological sensor 208 may be sent to the analysis component 210. The analysis component 210 may determine whether the subject's QT interval has shortened. If the shortened QT interval varies by more than a threshold (e.g., 10%, 20%, 30% and/or the like) from a baseline QT interval baseline subject, then the analysis component 210 may determine the subject to have low blood sugar. In embodiments, the analysis component 210 may be configured to determine a baseline QT interval of the subject when the subject is not experiencing low or high blood sugar.

Regarding nerve transit time, in embodiments, the physiological sensor 208 may be configured to stimulate one or more muscle fibers, sense (e.g., using an accelerometer) a muscle contraction in response to the stimulation and associate timings with both the stimulation of the muscle fiber and the contraction of the muscle fiber. One or more signals indicative of a timings associated with the stimulation and contraction of the muscle fibers may be sent to the analysis component 210. The analysis component 210 may determine a nerve transit time based on the timings associated with the stimulation and contraction of the muscle fibers. Further, the analysis component 210 may determine whether the nerve transit time is slower than a baseline nerve transit time for the subject by threshold (e.g., 10%, 20%, 30% and/or the like). In embodiments, the analysis component 210 may be configured to determine a baseline nerve transit time of the subject when the subject is not experiencing low or high blood sugar.

Regarding reflex sensitivity, in embodiments, the physiological sensor 208 may be configured to stimulate one or more muscle fibers and sense (e.g., using an accelerometer) the sensitivity (e.g., the amplitude) of a muscle contraction in response to the stimulation. One or more signals indicative of the amplitude of the response may be sent to the analysis component 210. The analysis component 210 may determine whether amplitude of the response is lower than a baseline response for the subject by threshold (e.g., 10%, 20%, 30% and/or the like). In embodiments, the analysis component 210 may be configured to determine a baseline reflex sensitivity of the subject when the subject is not experiencing low or high blood sugar.

Regarding autonomic tone, one or more signals indicative of a rate of firing of the sympathetic and/or parasympathetic systems (i.e., autonomic tone) may be sensed by the physiological sensor 208 and sent to the analysis component 210. The analysis component 210 may determine whether the subject's autonomic tone departs from a baseline by more than a threshold (e.g., 10%, 20%, 30% and/or the like) of the subject's normal autonomic tone, then the analysis component 210 may determine the subject to have low blood sugar. In embodiments, the analysis component 210 may be configured to determine a baseline autonomic tone of the subject when the subject is not experiencing low or high blood sugar.

As another example, the physiological sensor 208 may be configured to sense cognitive feedback (e.g., response given, response time and/or the like) to one or more cognitive tests. That is, it has been shown that a subject may become cognitively impaired including, for example, confusion and/or behavioral changes when a subject experiences low blood sugar (e.g., below 4.2 mmol/L). As such, the physiological sensor 208 may include an interface to present a subject with one or more cognitive tests and/or sense the responses to the one or more cognitive tests. One or more signals indicative of the responses may be sent to the analysis component 210. The analysis component 210 may then determine whether the responses (e.g., whether the responses given were correct) and/or one or more properties of the responses (e.g., time to respond, amount of questions answered and/or the like) depart from a baseline by more than a threshold (e.g., 10%, 20%, 30% and/or the like). In embodiments, the analysis component 210 may be configured to determine one or more cognitive feedback baselines for the subject when the subject is not experiencing low or high blood sugar.

In embodiments, the analysis component 210 may combine one or more of the examples provided above to determine an abnormal glycemic event and/or may combine one or more examples provided above with a determined surrogate compound presence and/or determined surrogate compound concentration. Further, in embodiments, in response to determining an abnormal glycemic event, an indication that an abnormal glycemic event was determined may be sent to the subject, an individual other than the subject (e.g., a spouse, caregiver, healthcare provider and/or the like) and/or another system (e.g., a healthcare monitoring system) so that corrective action may be taken. The indication may include, for example, sensory feedback such as: haptic, auditory and/or visual feedback indicative of the abnormal glycemic event.

In embodiments, the analysis component 210 may be implemented in any combination of hardware, software, and/or firmware, and may be implemented, at least in part, by the processor 212. In embodiments, the processor 212 may be, be similar to, include, or be included in, the processor 106 depicted in FIG. 1. The processor 212 may be any arrangement of electronic circuits, electronic components, processors, program components and/or the like configured to store and/or execute programming instructions, to direct the operation of the other functional components of the MD 200, for example, execute the instructions of the analysis component 210, and may be implemented, for example, in the form of any combination of hardware, software, and/or firmware.

In embodiments, the sensed emanated light may include one or more light pulses. As described above, a first pulse that is received by the analysis component 210 may transition the processor 212 from a lower-power state to a higher-power state. In embodiments, the processor 212 may be configured to be in a higher-power state to execute the instructions of the analysis component 210. Transitioning from a lower-power state to a higher-power state by the processor 212 may conserve power of the MD 200 and, therefore, may increase the longevity of the MD 200. Additionally or alternatively, the MD 200 may be configured to transition from a higher-power state to a lower-power state after executing the instructions of the analysis component 210.

The storage device 214 may be used to store information sensed by the optical sensor 206 and/or the physiological sensor 208 and/or determinations made by the analysis component 210 according to some implementations. The storage device 214 may include volatile and/or non-volatile memory, and may store instructions that, when executed by the MD 200 cause methods and processes to be performed by the MD 200. In embodiments, the processor 212 may process instructions and/or data stored in the storage device 214 to: control sensing and/or analysis operations performed by the MD 200, control communications performed by the MD 200, and/or the like.

While the light source 202, the optical sensor 206, the physiological sensor 208, the analysis component 210, the processor 212 and the storage device 214 are depicted as being incorporated into the MD 200, in embodiments, one or more of these components may be external to the MD 200. For example, the light source 202, the optical sensor 206, the physiological sensor 208, the analysis component 210, the processor 212 and/or the storage device 214 may be incorporated into a different MD (not shown). Alternatively, the light source 202, the optical sensor 206, the physiological sensor 208, the analysis component 210, the processor 212 and/or the storage device 214 may be located external to a subject. Additionally or alternatively, in embodiments, the light source 202, the optical sensor 206, the physiological sensor 208, the analysis component 210, the processor 212 and/or the storage device 214 may be distributed between multiple devices. That is, for example, the light source 202, the optical sensor 206, the physiological sensor 208, the analysis component 210, the processor 212 and/or the storage device 214 may refer to a number of different light sources, optical sensors, physiological sensors, analysis components, processors and/or storage devices each disposed on (and/or instantiated by) an MD or an external device.

The communication component 216 may include, for example, circuits, program components, and one or more transmitters and/or receivers for communicating non-wireless or wirelessly with one or more devices that are located external the MD 200 such as, for example, an external light source, an external optical sensor, an external physiological sensor, an external analysis component, an external processor and/or an external storage device. According to various embodiments, the communication component 216 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 216 may include any combination of hardware, software, and/or firmware configured to facilitate establishing, maintaining, and using any number of communication links. In embodiments, the communication component 216 may facilitate communications with other medical devices such as, for example, to facilitate coordinated operations between the medical devices.

In other embodiments, other forms of non-wireless or wireless telemetry may be utilized for communications. For example, in embodiments, other RF telemetry technologies may be employed. Alternatively, and/or additionally, inductive telemetry, acoustic telemetry and/or the like may be employed for communicating with, e.g., an external light source, an external optical sensor, an external analysis component, an external processor and/or an external storage device. In embodiments, conductive telemetry may be employed, in which case, for example, the communication component 216 may interact with one or more sensing/therapy electrode(s) to transmit and/or receive communications encoded in electrical pulses.

The power source 218 provides electrical power to the other operative components of the MD 200 (e.g., the light source 202, the optical sensor 206, the physiological sensor 208, the analysis component 210, the processor 212, the storage device 214 and/or the communication component 216) of the MD 200, and may be any type of power source suitable for providing the desired performance and/or longevity requirements of the MD 200. In various embodiments, the power source 218 may include one or more batteries, which may be rechargeable (e.g., using an external energy source). The power source 218 may include one or more capacitors, energy conversion mechanisms, and/or the like. Power sources for medical devices such as the MD 200 are well known, and are therefore not discussed in greater detail herein.

FIGS. 3A-3D are images of an illustrative indicator tag depicting different fluorescence responses to different surrogate compounds, in accordance with embodiments of the present disclosure. The indicator tag used in this embodiment was AcetonaPhthone phenyl ethyl Propionate Hydroxyl Tungstate (APPHT), which is response to hexane and/or ketones. That is, FIG. 3A depicts APPHT's response to hexane; FIG. 3B depicts APPHT's response to beta-hydroxybutyrate; FIG. 3C depicts APPHT's response to acetone; and FIG. 3D depicts APPHT unexposed to surrogate compound. Each of these compounds depicted in FIGS. 3A-3C are correlated to abnormal glycemic events when present in the interstitial fluid of a subject. As such, when an analysis component (e.g., the analysis component 210 depicted in FIG. 2) receives a signal from an optical sensor (e.g., the optical sensor 120 depicted in FIG. 1 and/or the optical sensor 206 depicted in FIG. 2) that corresponds to a sensed change in one of the fluorescence's depicted in FIG. 3A-3C, emanated from an indicator tag (e.g., the indicator tag 116 depicted in FIG. 1 and/or the indicator tag 204 depicted in FIG. 2), the analysis component may correlate the signal to the presence of a the respective surrogate compounds, i.e., hexane, beta-hydroxybutyrate and acetone. Based on the presence of the surrogate compound, the analysis component may determine the subject is experiencing an abnormal glycemic event. In embodiments, the analysis component may send an indication to the subject, an individual other than the subject (e.g., a spouse, caregiver, healthcare provider and/or the like) and/or another system (e.g., a healthcare monitoring system) that the analysis component determined that the subject is experiencing an abnormal glycemic event, so that can take corrective action (e.g., by administering an insulin injection if the subject is experiencing a hyperglycemic event or have sugar if the subject is experiencing a hypoglycemic event).

FIG. 4 depicts a graph of a fluorescence intensity change emanated from an indicator tag in response to different concentrations of a surrogate compound. The indicator tag used in this embodiment was AcetonaPhthone phenyl ethyl Propionate Hydroxyl and the surrogate compound was acetone. As shown, the fluorescence intensity changed linearly in response to changes in acetone concentration. Accordingly, in embodiments, when an analysis component (e.g., the analysis component 210 depicted in FIG. 2) receives a signal from an optical sensor (e.g., the optical sensor 120 depicted in FIG. 1 and/or the optical sensor 206 depicted in FIG. 2) that corresponds to a sensed fluorescence intensity, emanated from an indicator tag (e.g., the indicator tag 116 depicted in FIG. 1 and/or the indicator tag 204 depicted in FIG. 2) disposed in a subject, the analysis component may correlate the intensity to an acetone concentration that corresponds to the sensed intensity in the graph depicted in FIG. 4. In embodiments, the amount of acetone that is present in a subject when the subject is not experiencing an abnormal glycemic event, which may be a minimal amount, may be determined so that the analysis component can calibrate an amount of acetone that is present during normal glycemic events and levels of acetone that are indicative of abnormal glycemic events. In embodiments, the intensity vs. acetone concentration may be used in conjunction with the fluorescence depicted in FIG. 3C to determine the severity of the abnormal glycemic event.

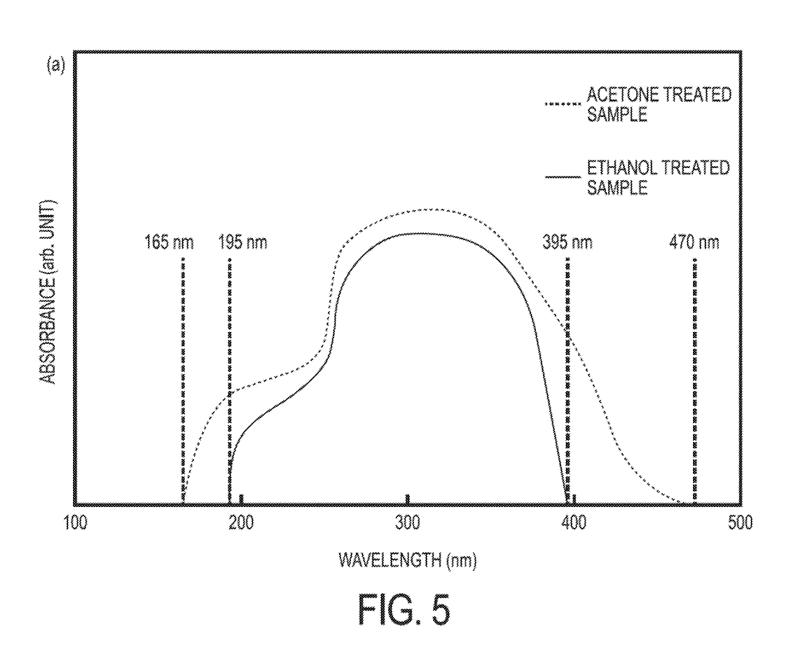

FIG. 5 is a graph depicting the varying light absorption of an acetone treated sample and an ethanol treated sample as a function of wavelength, in accordance with embodiments of the present disclosure. As shown, the light absorption of both the ethanol and the acetone treated samples vary in response to the wavelength of light to which the samples are exposed. Further, the acetone sample and the ethanol sample have similar absorption responses to the different wavelengths. For example, if the acetone and ethanol samples are exposed to light between 195 nanometers (nm) and 395 nm, both of the samples absorb some of the light to which they are exposed. As another example, both samples increase in their absorption from 195 nm until absorbing the most of amount of light when exposed to wavelengths that are approximately between 300 nm and 350 nm; after which, the absorption rates decrease. Accordingly, when an indicator tag (e.g., the indicator tag 116 depicted in FIG. 1 and/or the indicator tag 204 depicted in FIG. 2) that is responsive to acetone is exposed to acetone, the changed absorbance of the indicator tag may be correlated to a blood glucose concentration. As such, when an analysis component (e.g., the analysis component 210 depicted in FIG. 2) receives a signal from an optical sensor (e.g., the optical sensor 120 depicted in FIG. 1 and/or the optical sensor 206 depicted in FIG. 2) that corresponds to a sensed absorbance by an indicator tag (e.g., the indicator tag 116 depicted in FIG. 1 and/or the indicator tag 204 depicted in FIG. 2) that is responsive to acetone, the analysis component may correlate the absorbance to the presence of acetone in the subject. Further, the analysis component may correlate the presence of the acetone to an abnormal presence of sugar in the blood stream of the subject based on the correlation of acetone and ethanol depicted in FIG. 5. Based on the abnormal presence of sugar in the blood stream, the analysis component may determine the subject is experiencing an abnormal glycemic event. In embodiments, the analysis component may send an indication to the subject, an individual other than the subject (e.g., a spouse, caregiver, healthcare provider and/or the like) and/or another system (e.g., a healthcare monitoring system) that the analysis component determined that the subject is experiencing an abnormal glycemic event, so that the subject can take corrective action (e.g., by administering an insulin injection if the subject is experiencing a hyperglycemic event).

FIG. 6 is a graph depicting the delay in the varying light absorption of an indicator tag in response to a surrogate compound as a function of wavelength, in accordance with embodiments of the present disclosure. In this embodiment, the indicator tag is APPHT and the surrogate compound is acetone. As shown, the APPHT absorbs the greatest amount of light at approximately 368 nm. Further, after being exposed to acetone, the APPHT increases its absorption as a function of time. For example, the absorption of the APPHT from approximately 5 minutes to approximately 15 minutes after exposure. Accordingly, in embodiments, when an analysis component (e.g., the analysis component 210 depicted in FIG. 2) receives a signal from an optical sensor (e.g., the optical sensor 120 depicted in FIG. 1 and/or the optical sensor 206 depicted in FIG. 2) that corresponds to a sensed absorbance by an indicator tag (e.g., the indicator tag 116 depicted in FIG. 1 and/or the indicator tag 204 depicted in FIG. 2) disposed in a subject, the analysis component may correlate the sensed absorbance to the presence of acetone. Based on the presence of acetone, the analysis component may determine the subject is experiencing an abnormal glycemic event, as described above. Further, in embodiments, the analysis component may correlate the sensed absorbance to a time that the acetone has been present in the subject. In embodiments, this may give an indication for the severity of the abnormal glycemic event. In embodiments, the analysis component may send an indication to the subject, an individual other than the subject (e.g., a spouse, caregiver, healthcare provider and/or the like) and/or another system (e.g., a healthcare monitoring system) that the analysis component determined that the subject is experiencing an abnormal glycemic event and/or the severity of the abnormal glycemic event, so that the subject can take corrective action (e.g., by administering an insulin injection if the subject is experiencing a hyperglycemic event).

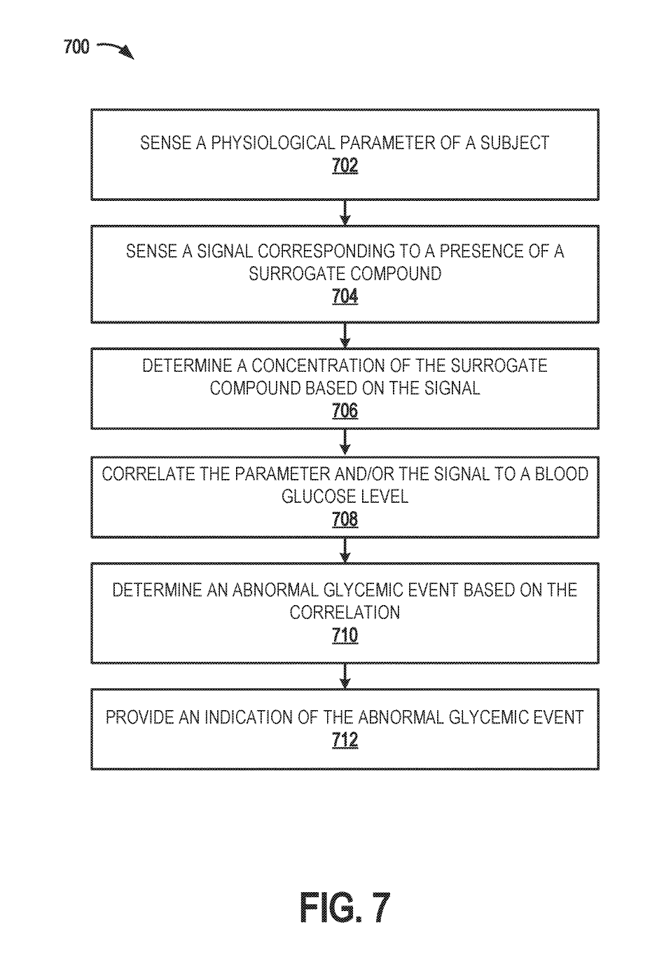

FIG. 7 is a flow diagram depicting an illustrative method 700 for determining an abnormal glycemic event using one or more surrogates for glucose, in accordance with embodiments of the present disclosure. In embodiments, the method 700 comprises sensing a physiological parameter of a subject (block 702). In embodiments, a medical device that may be, be similar to, include, or be included in, the medical device 102 depicted in FIG. 1 and/or the medical device 200 depicted in FIG. 2 may be used to sense the physiological parameter. In embodiments, the physiological parameter may be, be similar to, include, or be included in, the physiological parameters discuss above in relation to FIGS. 1 and 2. For example, the physiological parameter may include: an acceleration, heart rate variability, a QT interval, a nerve transit time, reflex sensitivity, an autonomic and feedback to a cognitive test.

In embodiments, the method 700 may further comprise sensing a signal corresponding to a presence of a surrogate compound and/or surrogate compound concentration (block 704). In embodiments, the surrogate compound and/or surrogate compound concentration may be a surrogate for glucose. In embodiments, the surrogate compound and/or surrogate compound concentration may be, be similar to, include, or be included in, the surrogate compounds and/or surrogate compound concentrations, respectively, discussed above in relation to FIGS. 1-6. For example, the compound may be hexane and/or a ketone (e.g., beta-hydroxybutyrate, acetone, acetoacetic acid).

In embodiments, a medical device used to sense the signal corresponding to the surrogate compound and/or surrogate compound concentration may be, be similar to, include, or be included in, the medical device 102 depicted in FIG. 1 and/or the medical device 200 depicted in FIG. 2. For example, sensing a signal corresponding to a presence of a surrogate compound may include sensing a signal in at least one of: an exhalation breath, interstitial fluid, blood and urine, wherein the compound is a surrogate for glucose. As another example, the medical device may be an implantable medical device and include an indicator tag that is exposed to interstitial fluid of the subject and responsive to the presence of the surrogate compound in interstitial fluid. Sensing a signal corresponding to a presence of the surrogate compound may include exposing the indicator tag to light and receiving emanated light from the indicator tag in response to the indicator tag being exposed to light. In this example, sensing a signal corresponding to a presence of a surrogate compound may include sensing an intensity of the light emanated from the indicator tag. Additionally or alternatively, sensing a signal corresponding to a presence of a surrogate compound may include fluorescence of the light emanated by the indicator tag. Additionally or alternatively, sensing a signal corresponding to a presence of a surrogate compound may include sensing a fluorescence lifetime effect of the indicator tag. In embodiments, the indicator tag may include AcetonaPhthone phenyl ethyl Propionate Hydroxyl Tungstate.

In embodiments, the method 700 may further comprise determining a concentration of the surrogate compound based on the signal (block 706). In embodiments, determining a concentration of the surrogate compound based on the signal may be, be similar to, include, or be included in, the embodiments discussed above in relation to FIGS. 2-6 for determining a concentration of the surrogate compound based on the signal. For example, the graph depicted in FIG. 4 may be used to determine an acetone concentration.

In embodiments, the method 700 may further comprise correlating the parameter and/or the signal to a blood glucose level (block 708). In embodiments, correlating the parameter and/or signal to a blood glucose level may be, be similar to, include, or be included in, the embodiments discussed above in relation to FIGS. 2-6 for correlating the parameter and/or the signal to a blood glucose level. For example, in embodiments, correlating the sensed parameter to a blood glucose level may include determining a ratio of the intensity and/or the frequency of the emanated light to the intensity and/or the frequency of the exposed light, wherein the ratio is indicative of a blood sugar level. As another example, the presence of the surrogate compound and/or concentration of the surrogate compound may indicate a specific blood sugar level. As even another example, correlating the parameter may include determining how much the parameter varies from a baseline.

In embodiments, the method 700 may further comprise determining an abnormal glycemic event based on the correlation (block 710). In embodiments, determining an abnormal glycemic event based on the correlation may be, be similar to, include, or be included in, the embodiments discussed above in relation to FIGS. 2-6 for determining an abnormal glycemic event based on the correlation.

In embodiments, the method 700 may further comprise providing an indication of the abnormal glycemic event to the subject, an individual other than the subject (e.g., a spouse, caregiver, healthcare provider and/or the like) and/or another system (e.g., a healthcare monitoring system) (block 712). In embodiments, providing an indication of the abnormal glycemic event to the subject, an individual other than the subject and/or another system may be, be similar to, include, or be included in, the embodiments discussed above in relation to FIGS. 2-6 for providing an indication of the abnormal glycemic event to the subject, the individual other than the subject and/or another system. For example, the indication may include sensory feedback such as: haptic, auditory and/or visual feedback indicative of the abnormal glycemic event.

Using various embodiments described herein, a glucose level of a subject may be determined using a surrogate for glucose. Based on the determined glucose level, one or more medical conditions may be determined and, in some cases, corrective action may be taken.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical system comprising:
a medical device associated with a subject, wherein the medical device is configured to sense a signal corresponding to a presence of a compound in at least one of: an exhalation breath, interstitial fluid, blood and urine, wherein the compound is a surrogate for glucose; and
a processor communicatively coupled to the medical device, the processor configured to:
receive the signal corresponding to the presence of the compound;
determine the presence of the compound based on the received signal; and
determine the subject is experiencing an abnormal glycemic event in response to the determined presence of the compound.

2. The medical system of claim 1,
wherein the medical device is an implantable medical device and comprises an indicator tag, wherein the indicator tag is responsive to the compound; and
wherein to sense a signal corresponding to the presence of the compound, the medical device is configured to sense light emanated from the indicator tag, wherein the light emanated from the indicator tag is in response to the indicator tag being exposed to light.

3. The medical system of claim 2, wherein to sense light emanated from the indicator tag, the medical device is configured to sense a fluorescence of the light emitted by the indicator tag.

4. The medical system of claim 2, wherein to sense light emanated from the indicator tag, the medical device is configured to sense a fluorescence lifetime effect of the indicator tag.

5. The medical system of claim 2, wherein the processor is configured to determine at least one of: a ratio of an intensity of the emanated light to an intensity of the exposed light and a ratio of a wavelength of the emanated light to a wavelength of the exposed light.

6. The medical system of claim 2,
wherein the exposed light comprises a first wavelength and a second wavelength and the emanated light comprises the first wavelength and the second wavelength; and
wherein the processor is configured to determine the presence of the compound based on the received signal by determining: a first absorption of the first wavelength by the indicator tag, a second absorption of the second wavelength by the indicator tag and comparing the first absorption to the second absorption.

7. The medical system of claim 2, wherein the processor is further configured to determine a concentration of the compound based on the received signal.

8. The medical system of claim 2, wherein the indicator tag comprises at least one of: a glucose-responsive fluorescent hydrogel and AcetonaPhthone phenyl ethyl Propionate Hydroxyl Tungstate.

9. The medical system of claim 1, wherein the compound is at least one of: hexane, ketones, catecholamine, cortisol, epinephrine and/or nor-epinephrine.

10. The medical system of claim 1, wherein the medical device is further configured to sense at least one of: the subject's acceleration, the subject's heart rate variability, a QT interval of the subject, a nerve transit time of the subject, the subject's reflex sensitivity, an autonomic tone of the subject and the subject's feedback to a cognitive test.

11. A method comprising:
sensing, using a medical device associated with a subject, a physiological parameter including at least one of: an acceleration, heart rate variability, a QT interval, a nerve transit time, reflex sensitivity, an autonomic and feedback to a cognitive test;
correlating the sensed parameter to a blood glucose level; and
determining, for the subject, using a processing device, an abnormal glycemic event based on the correlation of the sensed parameter to the blood glucose level.

12. The method of claim 11, further comprising sensing a signal corresponding to a presence of a compound in at least one: an exhalation breath, interstitial fluid, blood and urine, wherein the compound is a surrogate for glucose.

13. The method of claim 12, wherein the medical device is an implantable medical device and comprises an indicator tag exposed to interstitial fluid of the subject, wherein the indicator tag is responsive to the presence of the compound in interstitial fluid; wherein sensing a signal corresponding to a presence of a compound comprises exposing the indicator tag to light and receiving emanated light from the indicator tag in response to the indicator tag being exposed to light.

14. The method of claim 13, wherein sensing a signal corresponding to a presence of a compound comprises sensing a fluorescence of the light emanated by the indicator tag.

15. The method of claim 13, wherein sensing a signal corresponding to a presence of a compound comprises sensing a fluorescence lifetime effect of the indicator tag.

16. The method of claim 13, wherein correlating the sensed parameter to a blood glucose level comprises determining at least one of: a ratio of an intensity of the emanated light to an intensity of the exposed light and a ratio of a wavelength of the emanated light to a wavelength of the exposed light.

17. The method of claim 16, wherein the exposed light comprises a first wavelength and a second wavelength and the emanated light comprises the first wavelength and the second wavelength; and wherein determining a ratio of the emanated light to the exposed light comprises determining: a first absorption of the first wavelength by the indicator tag, a second absorption of the second wavelength by the indicator tag and comparing the first absorption to the second absorption.

18. The method of claim 13, further comprising determining a concentration of the compound based on the received emanated light.

19. The method of claim 12 wherein the indicator tag comprises at least one of: a glucose-responsive fluorescent hydrogel and AcetonaPhthone phenyl ethyl Propionate Hydroxyl Tungstate.

20. The method of claim 12, wherein the compound is at least one of: hexane, ketones, catecholamine, cortisol, epinephrine and/or nor-epinephrine.
